(19) United States
(12) Patent Application Publication
(10) Pub. No.: US 2011/0112466 A1
(43) Pub. Date: May 12, 2011

(54) Extended Point Phacoemulsification Tip

(76) Inventor: Ramon Carsola Dimalanta, Dove Canyon, CA (US)

(21) Appl. No.: 12/616,537

(22) Filed: Nov. 11, 2009

(51) Int. Cl. A61F 9/007

(52) U.S. Cl. 604/22

(57) ABSTRACT

In various embodiments, a phacoemulsification tip may be configured with a proximal end configured to be secured to a phacoemulsification hand piece and a distal end shaped as a five-sided polygon with five corners. In some embodiments, at least two of the five corners of the five-sided polygon may form a right angle and one of the five corners may form a dominant point spaced further from an axis of rotation of the five-sided polygon than any of the other four corners. For example, the distal end may be shaped in a home base configuration. The displacement of the dominant point from the axis of rotation may improve cutting and/or improve the removal of lens material. In some embodiments, the dominant point may form a sharper edge than at least one of the other four corners (e.g., the other four corners may be rounded).

### FIELD OF THE INVENTION

The present invention generally pertains to phacoemulsification. More particularly, but not by way of limitation, the present invention pertains to phacoemulsification cutting tips.

### DESCRIPTION OF THE RELATED ART

The human eye may provide vision by transmitting light through a clear outer portion called the cornea, and focusing the image by way of the lens onto the retina. The quality of the focused image may depend on many factors including the size and shape of the eye, and the transparency of the cornea and lens.

When age or disease causes the lens to become less transparent, vision may deteriorate because of the diminished light which can be transmitted to the retina. This deficiency in the lens of the eye may be referred to as a cataract. One treatment for this condition is surgical removal of the lens and replacement of the lens function by an intraocular lens (IOL).

Cataractous lenses may be removed by a surgical technique called phacoemulsification. During this procedure, a thin phacoemulsification cutting tip may be inserted into the diseased lens and vibrated ultrasonically. The vibrating cutting tip may liquefy or emulsify the lens so that the lens may be aspirated out of the eye. The diseased lens, once removed, may be replaced by an artificial lens (such as an IOL).

An ultrasonic surgical device suitable for ophthalmic procedures may include an ultrasonically driven hand piece, an attached cutting tip, an irrigating sleeve and an electronic control console. The hand piece assembly may be attached to the control console by an electric cable and flexible tubings. Through the electric cable, the console may vary the power level transmitted by the hand piece to the attached cutting tip and the flexible tubings may supply irrigation fluid to and draw aspiration fluid from the eye through the hand piece assembly.

The operative part of the hand piece may be centrally located, hollow resonating bar or horn directly attached to a set of piezoelectric crystals. The crystals may supply the required ultrasonic vibration needed to drive both the horn and the attached cutting tip during phacoemulsification and may be controlled by the console. The crystal/horn assembly may be suspended within the hollow body or shell of the hand piece by flexible mountings. The hand piece body may terminate in a reduced diameter portion or nosecone at the body's distal end. The nosecone may be externally threaded to accept the irrigation sleeve. Likewise, the horn bore may be internally threaded at its distal end to receive the external threads of the cutting tip. The irrigation sleeve also may have an internally threaded bore that is screwed onto the external threads of the nosecone. The cutting tip may be adjusted so that the tip projects only a predetermined amount past the open end of the irrigating sleeve.

In use, the ends of the cutting tip and irrigating sleeve may be inserted into a small incision of predetermined width in the cornea or sclera. The cutting tip may be ultrasonically vibrated along its longitudinal axis within the irrigating sleeve by the crystal-driven ultrasonic horn, thereby emulsifying the selected tissue in situ. The hollow bore of the cutting tip may communicate with the bore in the horn that in turn may communicate with the aspiration line from the hand piece to the console. A reduced pressure or vacuum source in the console may draw or aspirate the emulsified tissue from the eye through the open end of the cutting tip, the cutting tip and horn bores and the aspiration line and into a collection device. The aspiration of emulsified tissue may be aided by a saline flushing solution or irrigant that is injected into the surgical site through the small annular gap between the inside surface of the irrigating sleeve and the cutting tip.

### SUMMARY OF THE INVENTION

In various embodiments, a phacoemulsification tip may be configured with a proximal end configured to be secured to a phacoemulsification hand piece and a distal end shaped as a five-sided polygon with five corners. In some embodiments, at least two of the five corners of the five-sided polygon may form a right angle and one of the five corners may form a dominant point spaced further from an axis of rotation of the five-sided polygon than any of the other four corners. For example, the distal end may be shaped in a home base configuration. The displacement of the dominant point from the axis of rotation may improve cutting and/or improve the removal of lens material. In some embodiments, the dominant point may form a sharper edge than at least one of the other four corners (e.g., the other four corners may be rounded). In some embodiments, distal end may include a frame with the five-sided polygon shape and the interior of the five-sided polygon from the frame to an aspiration lumen may be hollow. Alternately, the distal end may include a solid structure with the five-sided polygon shape and the aspiration lumen may form an opening in the solid structure. Modifications to the home-base shape are also contemplated. For example, the distal end may include a home-base shape with two square portions removed from a bottom portion of the home-base shape above and on either side of the dominant point.

### BRIEF DESCRIPTION OF THE DRAWINGS

For a more complete understanding of the present invention, reference is made to the following description taken in conjunction with the accompanying drawings in which:

FIGS. 1a-b illustrate an ultrasound hand piece, according to an embodiment;

FIGS. 2a-g illustrate side and front views of a tip for the hand piece, according to an embodiment;

FIG. 3 illustrates a cross sectional view of the eye with a phacoemulsification tip inserted, according to an embodiment;

FIGS. 4a-b illustrate alternate embodiments of the tip; and

FIG. 5 illustrates a flowchart of an embodiment of a method for using the phacoemulsification tip to remove a lens.

It is to be understood that both the foregoing general description and the following detailed description are exemplary and explanatory only and are intended to provide a further explanation of the present invention as claimed.

### DETAILED DESCRIPTION OF THE EMBODIMENTS

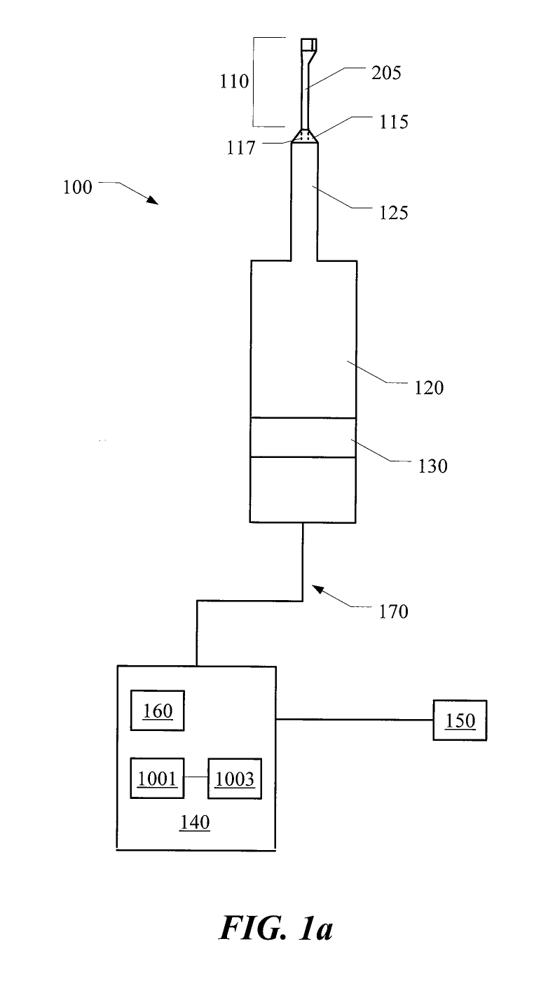

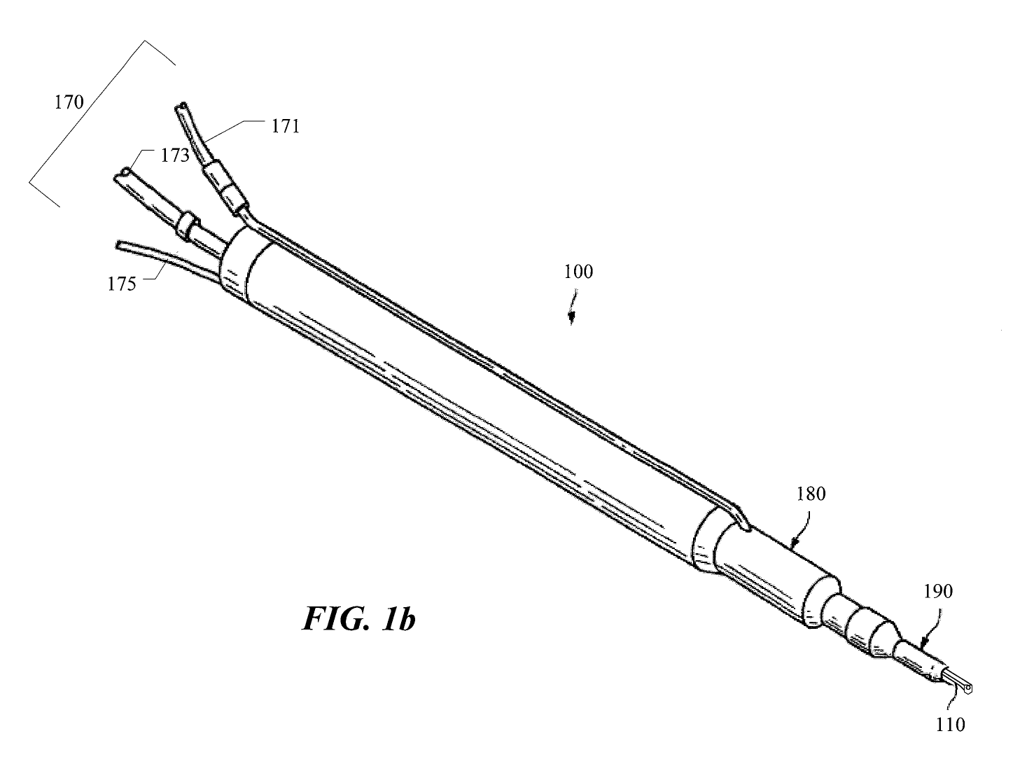

FIG. 1a illustrates an embodiment of an ultrasound hand piece 100. Hand piece 100 may be coupled to console 140. Console 140 may be coupled to an input device such as a foot switch 150. In some embodiments, hand piece 100 may include a cutting tip 110, a horn 120, and a set of piezoelectric crystals 130. A tip interface 115 may couple the cutting tip 110 to a reduced diameter portion 125 of horn 120 (e.g., a proximal end 117 of the cutting tip 110 may include threads configured to mate with threads on an interior of the tip interface 115). FIG. 1b illustrates hand piece 100 with an outer hand piece shell 180 and an irrigating sleeve 190. Other configurations of the outer hand piece shell and irrigating sleeve are also contemplated.

In some embodiments, the tip 110 may include a thin needle made of titanium or stainless steel (other materials are also contemplated) that is designed to emulsify a lens when vibrated ultrasonically. Tip 110 may include a cylindrical shaft 205 which may have a small diameter of about 20-30 gauge. In some embodiments, tip 110 may have a length suitable for removal of a lens when inserted into the anterior chamber of the eye.

Horn 120 may be made of a rigid material suitable for medical use (such as a titanium alloy). Horn 120 may include a reduced diameter section 125 that is connected to a tip interface 115. Tip interface 115 may include a threaded connection that accepts tip 110. In this manner tip 110 may be screwed onto horn 120 at tip interface 115. This may provide a rigid connection between tip 110 and horn 120 so that vibration can be transmitted from horn 120 to tip 110.

In some embodiments, piezoelectric crystals 130 may supply ultrasonic vibrations to drive both the horn 120 and the attached cutting tip 110 during phacoemulsification. Piezoelectric crystals 130 may be affixed to horn 120. Crystals 130 may be ring shaped, resembling a hollow cylinder and constructed from a plurality of crystal segments. Other crystal configurations are also contemplated. When excited by a signal from console 140, crystals 130 may resonate, producing vibration in horn 120. Tip 110, connected to horn 120, may also vibrate. When tip 110 is inserted into the anterior chamber of the eye and vibrated, it may act to emulsify a cataractous lens. Console 140 may include a signal generator 160 that produces the signal to drive piezoelectric crystals 130. Console 140 may also include a suitable microprocessor, micro-controller, computer, or digital logic controller (e.g., microprocessor 1001) to control the signal generator 160.

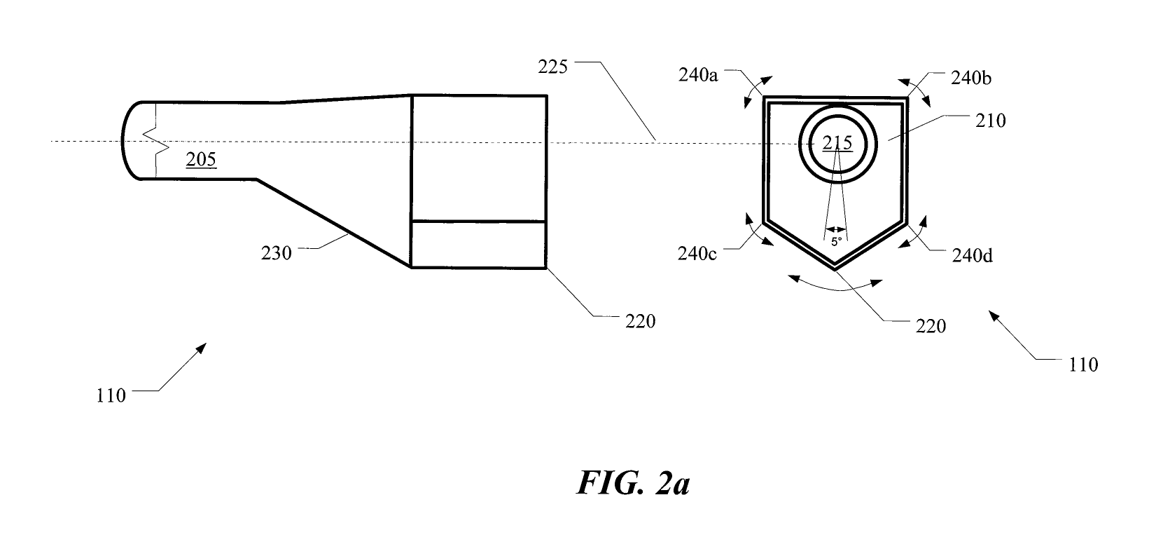

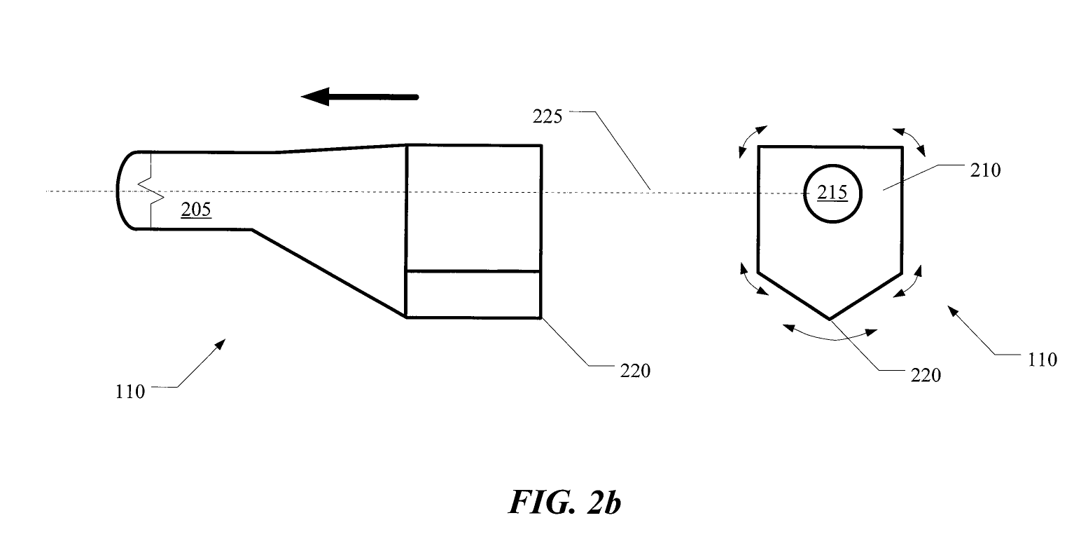

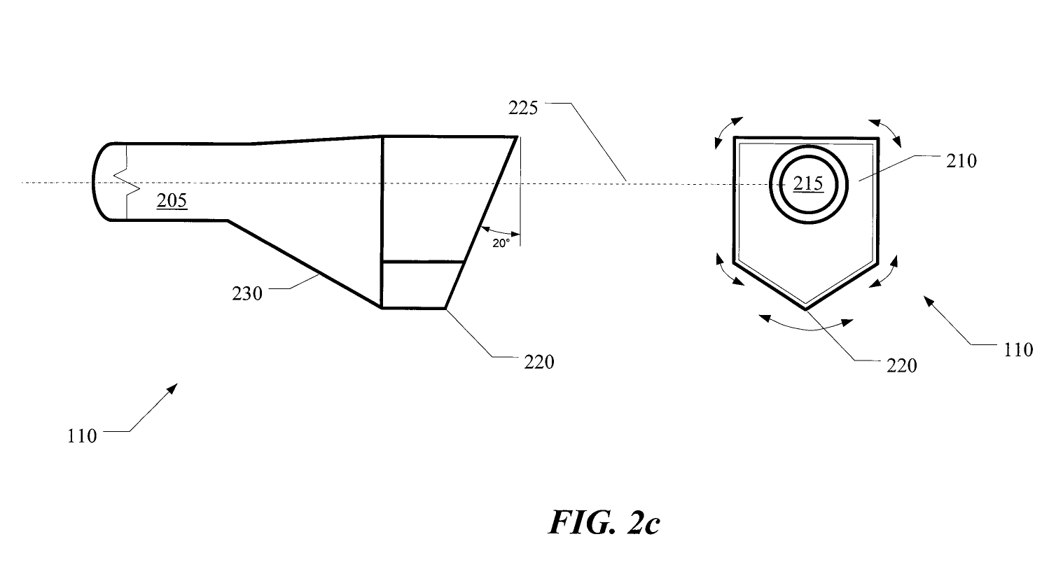

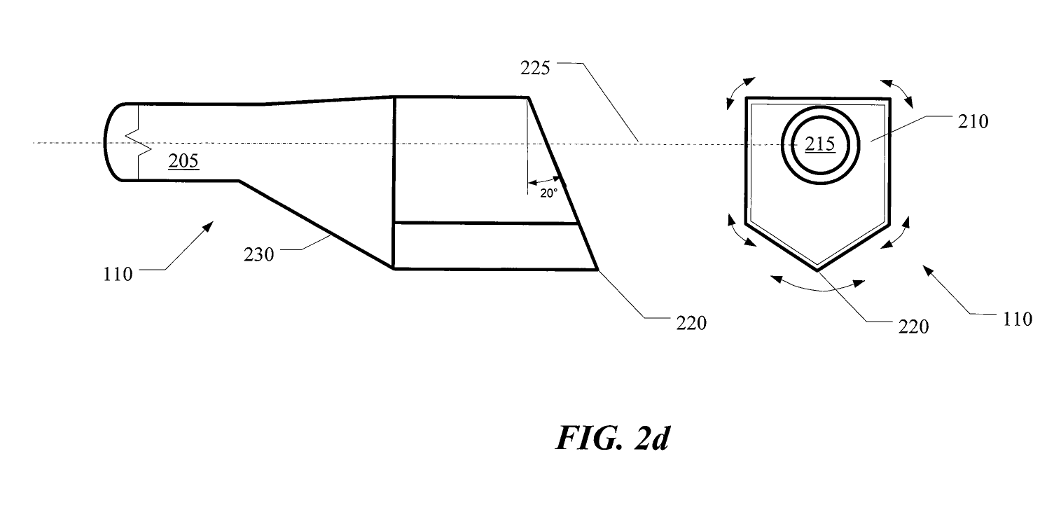

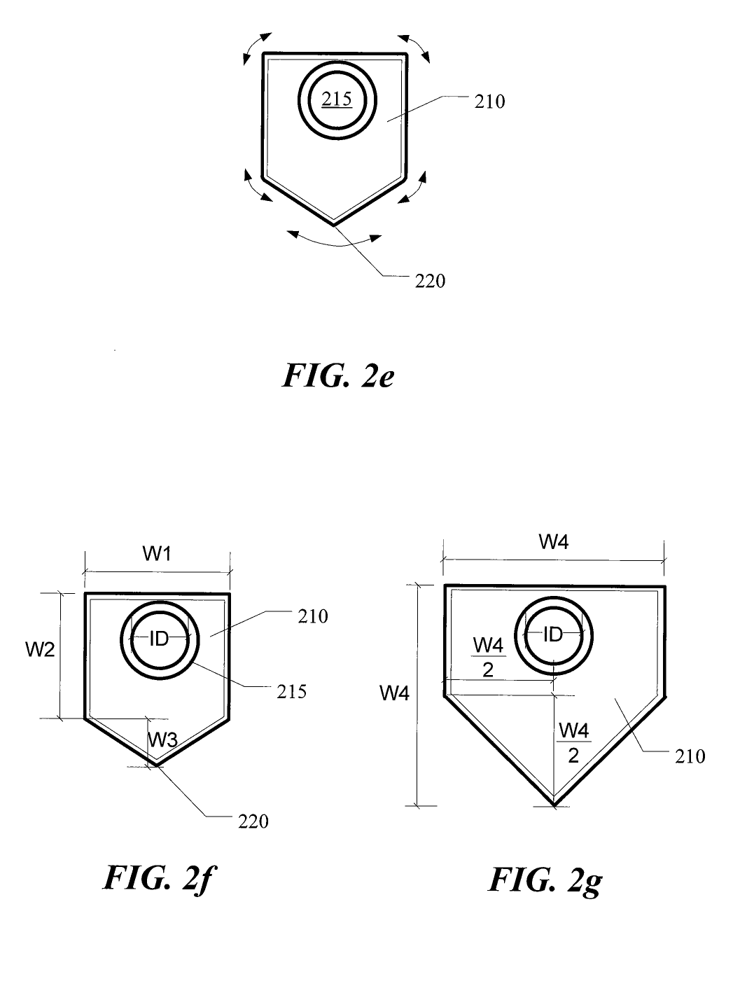

FIGS. 2a-g illustrate side and front views of embodiments of a tip 110 for the hand piece 100. The end of cutting tip 110 may be in the shape of an irregular polygon having a dominant point 220 (e.g., shaped similar to a baseball home-base) and a section 215 that forms an aspiration lumen. In some embodiments, a central aspiration lumen section 215 may be surrounded by section 210. As seen in FIG. 2a, section 210 may be hollow. As seen in FIG. 2b, section 210 may be solid. The arrow in FIG. 2b shows the direction of aspiration flow through the aspiration lumen. Lens material may be cut by the dominant point 220 of tip 110 when it is ultrasonically vibrated and aspirated through aspiration lumen section 215. The displacement of the dominant point 220 from the axis of rotation 225 may improve cutting and/or improve the removal of lens material. In some embodiments, the “home-base” shape may use ultrasound torsional movement (similar to a straight tip) to provide an arced motion of the dominant point 220 without having to use a bent tip (in some embodiments, a bent tip may be used). The displaced dominant point 220 may provide a torsional cutting edge displaced from the rotational axis 225. In some embodiments, the cutting tip 110 may be rotated back and forth on the rotational axis 225 along an angle of approximately 5 degrees. Other angles are also contemplated (e.g., between 3 and 10 degrees; between 10 to 40 degrees, etc). Other motion directions are also contemplated (e.g., longitudinal motion along the rotational axis). As seen in FIGS. 2a-b, the tip shaft 205 may gradually expand into the “home-base” shape through the expansion section 230. In some embodiments, the shaft 205 may expand primarily below the axis of rotation 225. However, in some embodiments, the shaft 205 may expand both above and below the axis of rotation 225. The eccentric placement of the dominant point 220 off the axis of rotation 225 may allow lateral movement at the far edges of the tip 110 without a bend in the shaft 205. In some embodiments, the “home-base” shape tip 110 may also be used with a bent shaft 205.

The “home-base” shape may include four points 240a-d that are approximately equidistant from the axis of rotation and one dominant point 220 placed further from the axis of rotation 225 than any of the other points 240a-d on the tip 110. In some embodiments, the axis of rotation 225 may be co-linear with a centerline of the cylindrical shaft 205. Other locations of the axis of rotation 225 are also contemplated. In some embodiments, at least two of the four corners 240a-d (e.g., the two top corners 240a-b) may form a substantially right angle (“substantially” including angles that are plus or minus 10 degrees from a 90 degree angle). The farthest placed point (the bottom of the “home-base” shape) may provide the most eccentric motion of the five points such that a surgeon can focus on placement of the dominant point 220 during the phacoemulsification procedure. The end-opening may be offset to allow the torsional movement of the shaft to translate into a side-to-side cutting edge. In some embodiments, the dominant point 220 may be sharper (e.g., come to a sharper angle) than the other four points 240a-d. For example, the other four points 240a-d may be rounded to make them duller than the dominant point 220 (e.g., as shown in FIG. 2e). In some embodiments, the other four points 240a-d may be as sharp or sharper than the dominant point 220.

FIGS. 2f-g illustrate some example dimensions. In some embodiments, the face of the tip 110 may include dimensions W1 approximately in a range of 0.027 to 0.05 inches; W2 approximately in a range of 0.027 to 0.05 inches; W3 approximately in a range of 0.01 to 0.025 inches; and inner diameter (ID) approximately in a range of 0.01 to 0.045 inches. Other dimensions and configurations are also contemplated. For example, as seen in the embodiment shown in FIG. 2g, dimension W4 may be approximately in a range of 0.035 to 0.07 inches and ID may be approximately in a range of 0.02 to 0.065 inches. In some embodiments, the axis of rotation 225 may be co-linear with a centerline of the aspiration lumen 215 such that the dominant point 220 is displaced further from the axis of rotation 225 than any other point on the tip face.

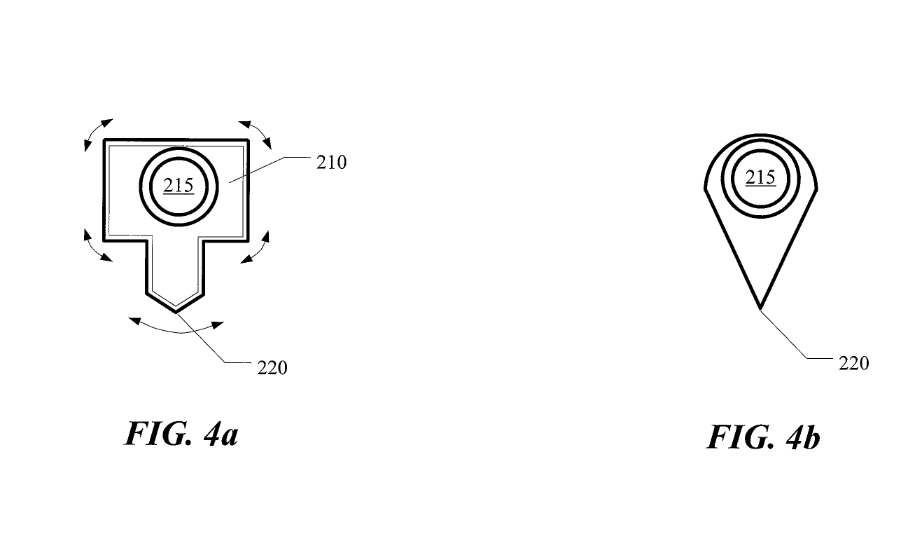

As seen in FIGS. 2c-d, in some embodiments, the tip 110 may be beveled (e.g., cut, molded, etc. at an angle). For example, an angle of 20 degrees may be used (other angles are also possible). In some embodiments, the tip 110 may not be beveled (e.g., as seen in FIGS. 2a-b). Additional embodiments are shown in FIGS. 4a-b. As seen in FIGS. 4a-b, the base “home-base” shape of tip may be modified by adding or subtracting material/shapes from the shape. For example, the “home-base” shape may have two rectangular portions removed from the bottom portion of the tip. In some embodiments, the four points 240a-d may be fully rounded such that the shape approaches a tear-drop shape as seen in FIG. 4b with dominant point 220. Other modifications are also contemplated.

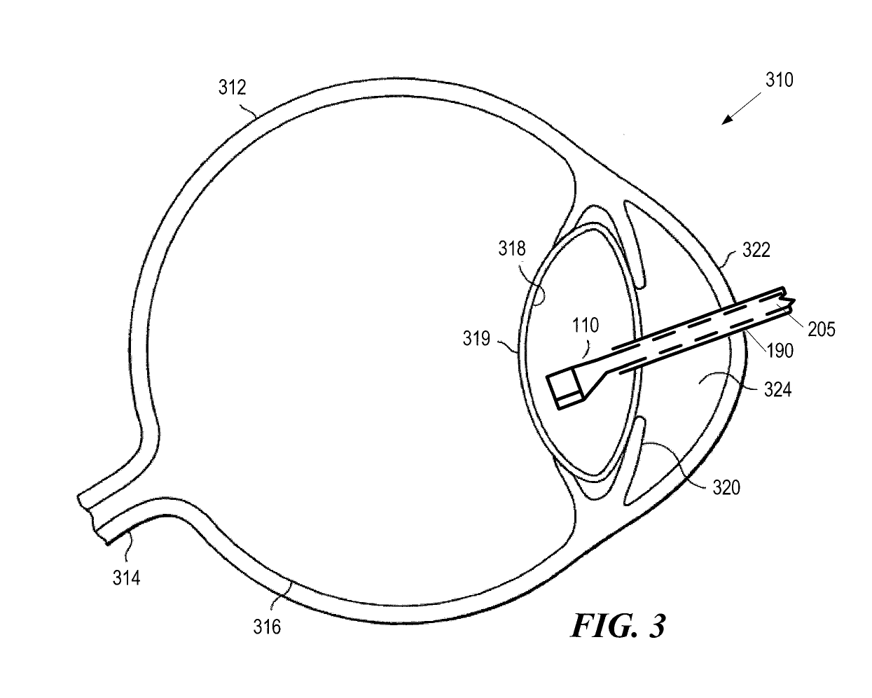

FIG. 3 illustrates a cross sectional view of eye 310 with phacoemulsification tip 110 inserted therein. Eye 310 may include sclera 312, optic nerve 314, retina 316, lens 318, capsular bag 319, iris 320, cornea 322, and pupil 324. Lens 318 may focus light passing through cornea 322 and pupil 324 onto retina 316. Retina 316 may convert light to nerve impulses which retina 316 may send along optic nerve 314 to the brain. Iris 320 may regulate the amount of light passing through pupil 324 and lens 318 thereby allowing eye 310 to adapt to varying levels of light. Capsular bag 319 may hold lens 318 in place and may be transparent so that light may pass through it. Thus, the nerve impulses traveling along optic nerve 314 may correspond to scenes visible to eye 310.

However, various diseases, conditions, injuries, etc. can cause lens 318 to become clouded, translucent, etc. to the point that it might be desirable to extract lens 318 from eye 310. In such situations, the affected patient may be said to have a “cataract.” When lens 318 is removed from eye 310 (i.e., the cataract is extracted), surgical personnel may replace lens 318 with an artificial lens, thereby restoring sight to the affected patient. Alcon Laboratories, Inc. (of Fort Worth, Tex.) provides exemplary artificial lenses such as the AcrySof® intraocular lenses. To remove lens 318, surgical personnel may use a hand piece 100 with phacoemulsification tip 110. As illustrated in FIGS. 1a-b, hand piece 100 may include tip 110 and may be connected to console 140 through connections 170 (which may include ophthalmic tubing 171 to provide irrigating fluid for irrigating sleeve 190, ophthalmic tubing 173 to return material aspirated from eye 310 to the console 140, and an electrical cable 175 for ultrasonic control/power). Hand piece 100 may provide fluid channels between the ophthalmic tubing 171 and the irrigating sleeve 190 on the tip 110. Additionally, hand piece 100 may couple with the irrigating sleeve 190 and indirectly with tip 110 (via one or more internal components) thereby holding these components 190 and 110 in fixed operational relationship to each other (such that the tip 110 can be vibrated independently of the sleeve 190 (which may be held stationary relative to the tip 110)).

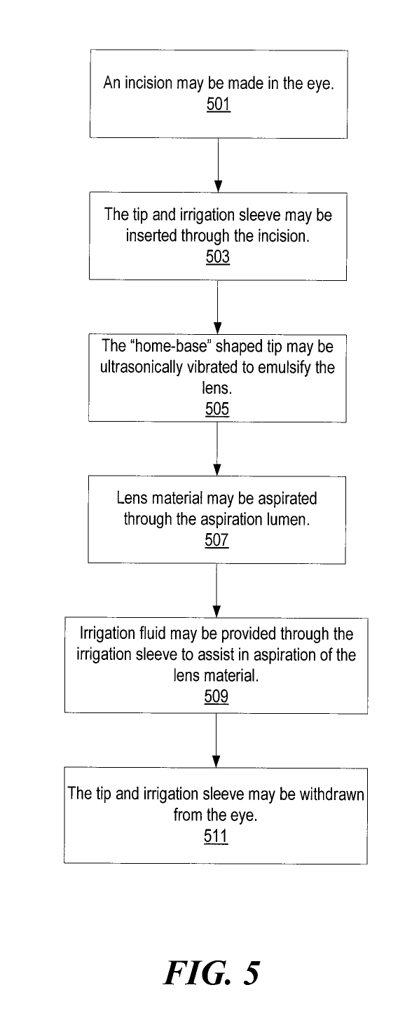

FIG. 5 illustrates a flowchart of an embodiment of a method for using the phacoemulsification tip to remove a lens. The elements provided in the flowchart are illustrative only. Various provided elements may be omitted, additional elements may be added, and/or various elements may be performed in a different order than provided below.

At 501, an incision may be made in the eye. For example, a surgical knife may be used to make an incision through the cornea 322 and to the capsular bag 319 to access the lens 318.

At 503, the tip 110 and irrigation sleeve 190 may be inserted through the incision and into contact with the lens 318.

At 505, a “home-base” shaped tip 110 may be ultrasonically vibrated to emulsify the lens 318. The displaced dominant point 220 in tip 110 may provide a torsional cutting edge displaced from the rotational axis 225. The displacement of the dominant point 220 in the tip 110 from the axis of rotation 225 may improve cutting and/or improve the removal of lens material.

At 507, as the tip 110 is emulsifying the lens 318, lens material may be aspirated through the aspiration lumen 215.

At 509, irrigation fluid may be provided through the irrigation sleeve 190 to assist in aspiration of the lens material.

At 511, the tip 110 and irrigation sleeve 190 may be withdrawn from the eye.

In some embodiments, the console 140 may include one or more processors (e.g., processor 1001). The processor 1001 may include single processing devices or a plurality of processing devices. Such a processing device may be a microprocessor, controller (which may be a micro-controller), digital signal processor, microcomputer, central processing unit, field programmable gate array, programmable logic device, state machine, logic circuitry, control circuitry, analog circuitry, digital circuitry, and/or any device that manipulates signals (analog and/or digital) based on operational instructions. The memory 1003 coupled to and/or embedded in the processors 1001 may be a single memory device or a plurality of memory devices. Such a memory device may be a read-only memory, random access memory, volatile memory, non-volatile memory, static memory, dynamic memory, flash memory, cache memory, and/or any device that stores digital information. Note that when the processors 1001 implement one or more of its functions via a state machine, analog circuitry, digital circuitry, and/or logic circuitry, the memory 1003 storing the corresponding operational instructions may be embedded within, or external to, the circuitry comprising the state machine, analog circuitry, digital circuitry, and/or logic circuitry. The memory 1003 may store, and the processor 1001 may execute, operational instructions corresponding to at least some of the elements illustrated and described in association with FIG. 5.

Various modifications may be made to the presented embodiments by a person of ordinary skill in the art. Other embodiments of the present invention will be apparent to those skilled in the art from consideration of the present specification and practice of the present invention disclosed herein. It is intended that the present specification and examples be considered as exemplary only with a true scope and spirit of the invention being indicated by the following claims and equivalents thereof.

What is claimed is:

1. A phacoemulsification tip, comprising:
a proximal end configured to be secured to a phacoemulsification hand piece; and
a distal end shaped as a five-sided polygon with five corners, at least two of the five corners forming a substantially right angle and wherein one of the five corners forms a dominant point spaced further from an axis of rotation of the phacoemulsification tip than any of the other four corners.

2. The phacoemulsification tip of claim 1, wherein the distal end is shaped in a home base configuration.

3. The phacoemulsification tip of claim 1, wherein the dominant point forms a sharper edge than at least one of the other four corners.

4. The phacoemulsification tip of claim 1, wherein the dominant point forms a sharp edge and wherein the other four corners are rounded.

5. The phacoemulsification tip of claim 1, wherein the distal end comprises a frame with the five-sided polygon shape and wherein the interior of the five-sided polygon from the frame to an aspiration lumen is hollow.

6. The phacoemulsification tip of claim 1, wherein the distal end comprises a solid structure with the five-sided polygon shape and wherein an aspiration lumen forms an opening in the solid structure.

7. The phacoemulsification tip of claim 1, wherein a shaft of the tip leading up to the distal end is cylindrical, wherein the distal end of the tip includes at least one dimension that is larger than a diameter of the shaft, and wherein the tip includes a transition region between the cylindrical shaft and the distal end.

8. The phacoemulsification tip of claim 1, wherein the distal end has a home-base shape with two rectangular portions removed from a portion of the home-base shape above and on either side of the dominant point.

9. An ophthalmic surgical handpiece, comprising:
a horn;
a set of piezoelectric crystals, coupled to the horn, configured to provide ultrasonic vibration to drive the horn; and
a phacoemulsification cutting tip coupled to the horn and configured to be ultrasonically vibrated by the horn, wherein the phacoemulsification tip comprises:
a proximal end configured to be secured to a phacoemulsification hand piece;
a distal end shaped to include a dominant point spaced further from an axis of rotation of the phacoemulsification tip than any other point on the distal end.

10. The ophthalmic surgical handpiece of claim 9, wherein the distal end is shaped in a home base configuration with a five-sided polygon having five corners, at least two of the five corners forming a substantially right angle and wherein one of the five corners forms the dominant point.

11. The ophthalmic surgical handpiece of claim 10, wherein the dominant point forms a sharper edge than at least one of the other four corners.

12. The ophthalmic surgical handpiece of claim 9, wherein the distal end is shaped as a tear-drop having a rounded end opposite the dominant point.

13. The ophthalmic surgical handpiece of claim 9, wherein the distal end comprises a frame with the five-sided polygon shape and wherein the interior of the five-sided polygon from the frame to an aspiration lumen is hollow.

14. The ophthalmic surgical handpiece of claim 9, wherein the distal end comprises a solid structure with the five-sided polygon shape and wherein an aspiration lumen forms an opening in the solid structure.

15. The ophthalmic surgical handpiece of claim 9, further comprising an irrigation sleeve coupled to the handpiece and configured to direct irrigation fluid to a portion of the eye interacting with the phacoemulsification tip.

16. The ophthalmic surgical handpiece of claim 9, wherein a shaft of the tip leading up to the distal end is cylindrical, wherein the distal end of the tip includes at least one dimension that is larger than a diameter of the shaft, and wherein the tip includes a transition region between the cylindrical shaft and the distal end.

17. The ophthalmic surgical handpiece of claim 9, wherein the distal end has a home-base shape with two rectangular portions removed from a portion of the home-base shape above and on either side of the dominant point.

18. A method, comprising:
inserting a phacoemulsification tip into an eye, wherein the phacoemulsification tip comprises a proximal end configured to be secured to a phacoemulsification hand piece and a distal end shaped as a five-sided polygon with five corners, at least two of the five corners forming a substantially right angle and wherein one of the five corners forms a dominant point spaced further from an axis of rotation of the phacoemulsification tip than any of the other four corners;
ultrasonically vibrating the phacoemulsification tip in the eye, wherein the vibration results in a torsional movement of the dominant point to emulsify a lens in the eye; and
aspirating lens material from the emulsified lens through an aspiration lumen in fluid communication with the phacoemulsification tip.

19. The method of claim 18, further comprising making an incision in the eye with a surgical knife prior to insertion of the phacoemulsification tip.

20. The method of claim 18, further comprising providing irrigation fluid through an irrigation sleeve configured to direct irrigation fluid to the eye at a site of lens emulsification.
